(19) United States
(12) Patent Application Publication
(10) Pub. No.: US 2009/0149832 A1
(43) Pub. Date: Jun. 11, 2009

(54) APPARATUS AND METHODS FOR FLUID TRANSFER VIA SUBCUTANEOUS PORT

(76) Inventors: David J. Sauvageau, Methuen, MA (US); Robert F. Rioux, Ashland, MA (US); Mark E. Girard, Medway, MA (US)

Correspondence Address: FAY KAPLUN & MARCIN, LLP, 150 BROADWAY, SUITE 702, NEW YORK, NY, 10038, US

(21) Appl. No.: 12/209,495

(22) Filed: Sep. 12, 2008

Related U.S. Application Data
(60) Provisional application No. 60/972,178, filed on Sep. 13, 2007.

(51) Int. Cl. A61M 5/31

(52) U.S. Cl. 604/513; 604/164.03

(57) ABSTRACT

A device for providing access to an implantable fluid transfer port, comprises a needle extending to a distal end insertable into body to enter a reservoir of a port implanted therein and an actuation member extending through a lumen of the needle from an actuator which remains external to a living body accessible to a user thereof in combination with an obturator coupled to a distal end of the actuation member, an outer diameter of the obturator substantially matching an inner diameter of the lumen of the needle so that, when retracted into the lumen, the obturator seals a distal opening thereof and an anchoring mechanism coupled to the actuation member proximally of the obturator, the anchoring mechanism being moved to an expanded state in which an outer diameter of the anchoring mechanism exceeds an outer diameter of the needle when moved out of the lumen of the needle.

### PRIORITY CLAIM

This application claims priority to U.S. Provisional Application Ser. No. 60/972,178 entitled “Apparatus and Methods for Injecting Fluid Through Subcutaneous Port,” filed Sep. 13, 2007. The entire disclosure of the above-identified application is incorporated by reference into this application.

### FIELD OF THE INVENTION

The present invention relates generally to apparatus and methods for delivering fluids to subcutaneous locations and more particularly, to apparatus and methods for injecting fluids through subcutaneous ports.

### BACKGROUND INFORMATION

Central venous system access is required for the long-term administration of fluids to (e.g., drugs, therapeutic agents, chemotherapy agents) and to draw blood or other fluids from a patient. For example, many medical procedures require that a patient receive an infusion that last for hours or even days. Infusions may also need to be repeated periodically over a period of months or years. Such long term access to the venous system is often obtained via a subcutaneous access port. Subcutaneous port systems often include an aperture sealed with a self-sealing septum for receiving a needle and an outlet connection leading to a transfer device, such as an implanted cannula or catheter, which may be fed into a vein or artery. A reservoir receiving the infused or withdrawn fluids is situated between the septum and the outlet connection. The subcutaneous port is accessed by inserting the needle through the skin of the patient and through the septum into the reservoir. A fluid may then be then injected through the needle into the reservoir or withdrawn from the body into the needle as would be understood by those skilled in the art.

Multiple punctures applied to the septum over the long-term period of use may cause the septum to wear out and thus be unable to provide a fluid tight seal to the reservoir. Thus, fluids may leak from the reservoir and into the surrounding tissue, thus causing potential harm thereto. Additionally, long-term use of the septum may cause a needle used therewith to core out or dislodge small particles of the seal with each penetration, thus creating a permanent channel through the septum over an extended period. Still further, a needle may be prematurely removed from the septum under power injection treatment. In each case, harmful or painful agent may be injected under the patient's skin.

### SUMMARY OF THE INVENTION

A device for providing access to an implantable fluid port, comprises a needle extending from a proximal end located external to a living body to a distal end insertable into a reservoir of the implantable port via a septum provided thereover, wherein a cannula extends through the needle from a proximal end open to an inlet port to a distal opening. A probe is slidably disposed within the cannula, the probe having an elongated probe shaft and further comprising an obturator located at a distal end of the probe shaft, wherein an outer diameter of a portion of the obturator is sufficient to seal the distal opening of the cannula and an expanding anchoring mechanism located along a distal length of the probe shaft. The needle is movable between an insertion configuration where the obturator seals the distal opening of the cannula and an expanded configuration wherein the obturator is moved distally so that the anchoring mechanism is moved distally of the distal end of the needle, wherein distal movement of the probe causes the anchoring mechanism to move to the are deployed distally of a distal opening of the needle.

The present invention is directed to a device for providing access to an implantable fluid transfer port, comprising a needle extending to a distal end insertable into a living body to enter a reservoir of a port implanted therein and an actuation member extending through a lumen of the needle from an actuator which remains external to a living body accessible to a user thereof in combination with an obturator coupled to a distal end of the actuation member, an outer diameter of the obturator substantially matching an inner diameter of the lumen of the needle so that, when retracted into the lumen, the obturator seals a distal opening thereof and an anchoring mechanism coupled to the actuation member proximally of the obturator, the anchoring mechanism being moved to an expanded state in which an outer diameter of the anchoring mechanism exceeds an outer diameter of the needle when moved out of the lumen of the needle.

### BRIEF DESCRIPTION OF DRAWINGS

FIG. 1 is a plan view of a medical system for delivering fluid to a patient according to an exemplary embodiment of the present invention;

FIG. 2 shows a top view of a subcutaneous port of the system of FIG. 1;

FIG. 3 shows a partial cross-sectional view of the subcutaneous port of FIG. 2 taken along the line 3-3;

FIG. 4A shows a first side view of an exemplary needle according to the present invention in a first retracted configuration;

FIG. 4B shows a second side view of the needle of FIG. 4A in a deployed configuration;

FIG. 4C shows a cross sectional view of the needle FIGS. 4A and 4B, taken along the line 4C-4C;

FIG. 5A shows a partial cut-through view of a system according to the present invention in a retracted configuration in a subcutaneous port;

FIG. 5B shows a partial cut-through view of the system of FIG. 5A in a deployed configuration;

FIG. 5C shows a partial cut-through view of the system of FIG. 5A once returned to the retracted configuration; and

FIG. 5D shows the system of FIG. 5A with the needle removed from the subcutaneous port.

### DETAILED DESCRIPTION

The present invention may be further understood with reference to the following description and to the appended drawings, wherein like elements are referred to with the same reference numerals. The present invention relates to devices for providing long-term access to subcutaneously implanted access ports for infusing or withdrawing fluids from a living body. Specifically, a system and method according to the present invention seeks to minimize damage to a septum of a subcutaneous port with long-term use. It is noted that, although embodiment of the present invention are directed to septa of subcutaneous ports, the present invention may be employed in any port that utilizes a septum over an inlet port thereof.

A system according to the present invention comprises an implantable fluid transfer assembly and a fluid injection needle. The fluid transfer assembly comprises a reservoir for into which fluids may be injected and/or into which fluids may be drawn from the body before entering the needle. The assembly also includes an outlet port in fluid communication with the reservoir and an inlet port with a self-sealing septum in fluid communication with the reservoir. In one embodiment, the fluid transfer assembly comprises a subcutaneous port that carries the reservoir, inlet port, and outlet port, and a catheter in fluid communication with the outlet port of the subcutaneous port. The fluid injection needle comprises a cannula formed as an elongated shaft with a lumen extending therethrough and a distal outlet port and an inner probe slidably disposed within the lumen of the cannula. In one embodiment, the cannula has a fluid infusion port in fluid communication with the cannula lumen, and the medical system further comprises a pump in fluid communication with the fluid infusion port. The inner probe includes an elongated shaft, an obturator carried on an inner probe shaft thereof and a self-expanding anchoring mechanism carried by the inner probe shaft. In one embodiment, the self-anchoring mechanism is a resilient basket structure. In another embodiment, the obturator is disposed distal to the anchoring mechanism and comprises a cylindrical member and an obturator tip (e.g., a spherically-shaped tip). The fluid injection needle is configured to be moved between an insertion/removal state with the anchoring mechanism retracted within the cannula lumen and the obturator seated within the outlet port of the cannula lumen, and a fluid delivery/withdrawal state with the obturator and anchoring mechanism deployed from the cannula shaft.

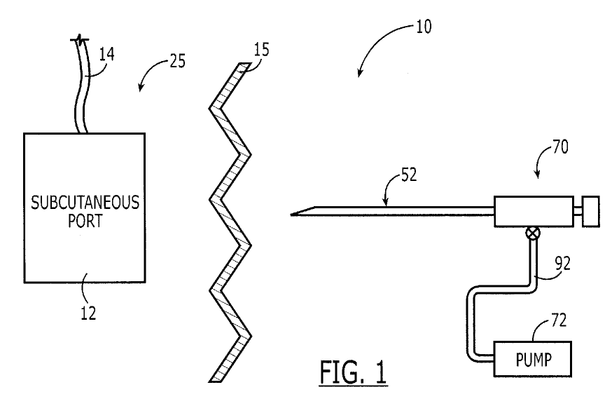

As shown in FIG. 1, an implantable fluid transfer system 10 according to the invention comprises an implantable fluid transfer assembly 25 including a subcutaneous port 12 and a catheter 14 in fluid communication with the subcutaneous port 12 for periodically administering fluids (e.g., for nutrition, hydration, drugs and/or other therapeutic agents, etc.) to or withdrawing fluids from a designated vessel (e.g., a blood vessel). The fluid transfer system 10 further comprises an external fluid transfer apparatus 70 for delivering fluids (e.g., therapeutic agents) to the vessel and/or withdrawing fluids therefrom via the implantable fluid transfer assembly 25, and in particular, into the subcutaneous port 12 thereof. As described above, fluids travel between the vessel and the external fluid transfer apparatus 70 via the catheter 14, the subcutaneous port 12 and a needle 52 of the external fluid transfer apparatus 70. As indicated above, the therapeutic agent may comprise hydrating fluids, nutrition, drugs, biologics, proteins, genetic materials, or any other substance or material which medical personnel may prescribe as beneficial for the patient, as those skilled in the art will understand.

As known to those skilled in the art, the subcutaneous port 12 may be implanted, for example, just below a target portion of the skin 15 adjacent a in an area selected for its ease of access and/or proximity to a targeted blood vessel, etc. and the catheter 14 is subcutaneously tunneled to the targeted blood vessel. The external fluid transfer apparatus 70 may be employed to supply fluids to the subcutaneous port 12 in accordance with a method explained in detail hereinafter. The external fluid transfer apparatus 70 generally comprises a needle 52 connected to an external power injection pump 72 via a tube 92 formed of a material selected as would be understood by those skilled in the art to be suitably flexible and resilient to enable it to operate during high-pressure power injections. The subcutaneous port 12 of the present invention is also suitable for use at the relatively high pressures associated with power injections induced by the external fluid transfer apparatus 70 without leakage or failure. Moreover, the subcutaneous port 12 is suitable for repeated use at the pressures induced by the external fluid transfer apparatus 70. In one aspect, for example, the subcutaneous port 12 is suitable to withstand the pressure of at least approximately 300 psi generated by the external power injection pump 72 without leakage or failure.

The external fluid transfer apparatus 70 may be operated over a wide variety of flow rates and working pressures. In the illustrated embodiment, the external fluid transfer apparatus 70 injects fluid into the subcutaneous port 12 at a flow rate equal to or greater than 5 cc/sec. It is noted that, although embodiments of the present invention are described with respect to a high pressure external fluid transfer apparatus 70, any commercially available power injection apparatus, such as rotary pumps, in-line pumps and gear pumps, is suitable for use with the system 10 of the present invention. Furthermore, in an alternate embodiment, a manually actuated injector pump (not shown) may be used in place of the power injection pump 72.

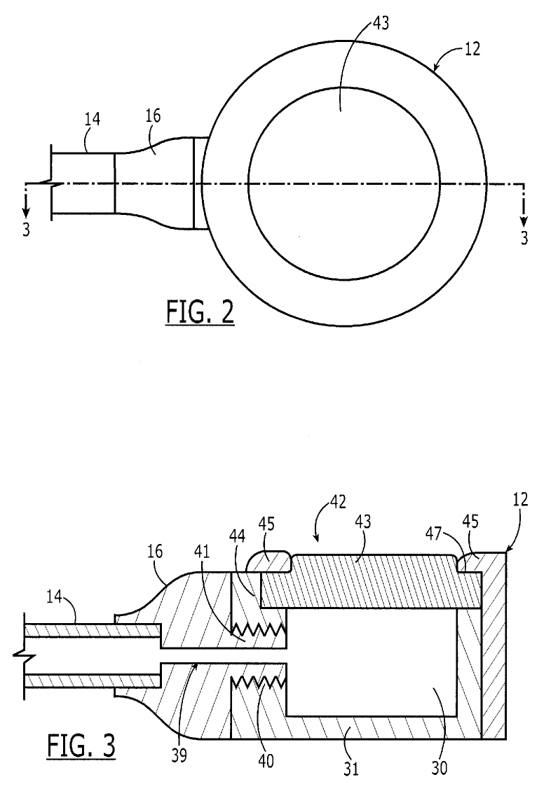

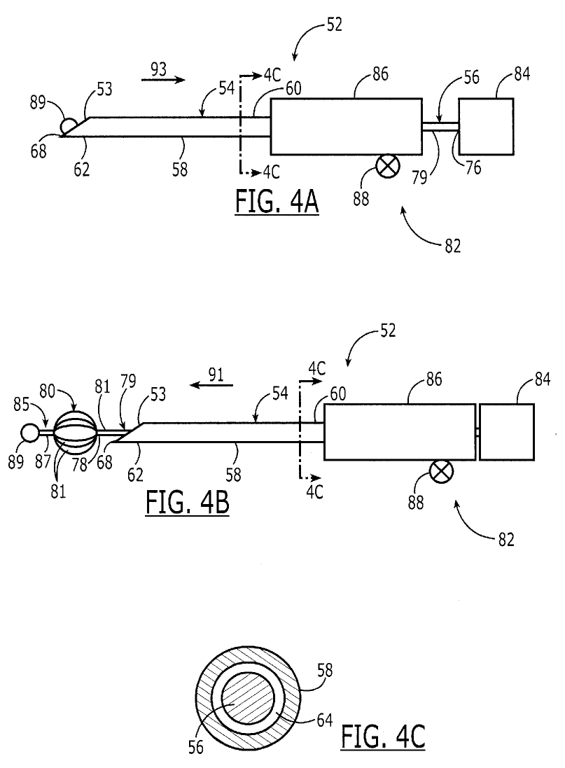

As shown in FIGS. 2 and 3, the subcutaneous port 12 of the present invention comprises a housing 31 formed of a biocompatible material, such as stainless steel or a titanium alloy, and is of a size and shape that facilitates implantation thereof under the skin 15 of the patient. In an exemplary embodiment, the shape of the subcutaneous port 12 comprises a relatively flat housing 31 having rounded corners to avoid undue trauma to adjacent tissue when implanted in situ much like existing implantable pacemakers or cochlear stimulators. The subcutaneous port 12 further comprises a reservoir 30 for housing a therapeutic agent therein. At a distal end, the reservoir 30 is in fluid communication with an outlet port 39 which is in further fluid communication with the catheter 14 connected to a blood vessel of the patient. The catheter 14 includes an adapter 16 that can be releasably mated with the outlet port 39 of the subcutaneous port 12. As shown in greater detail in FIG. 3, the housing 31 of the subcutaneous port 12 comprises a threaded female portion 40 located adjacent to the outlet port 39. A respectively threaded male portion 41 of the adapter 16 is sized and shaped to permit alignment of the outlet port 39 with the catheter 14 when the threaded male portion 41 is screwed in the female portion 40. In this manner, a fluid-tight engagement is forged between the subcutaneous port 12 and the catheter 14.

At a proximal end, the reservoir 30 is open to an inlet port 42 with a self-sealing septum 43 formed thereover. The septum 43 is formed as a substantially circular member with an annular septum recess 47 formed along an outer perimeter thereof. It is noted, however, that the septum 43 may assume any shape known in the art such as, for example, a rectangle, wherein a shape of the housing may be chosen to accommodate the shape of the septum 43. Furthermore, the septum 43 may be formed of a material suitable for subcutaneous implantation, as known to those of skill in the art. The septum 43 is fluidly sealed against the inlet port 42 to prevent a leakage of fluid from a perimeter thereof. Specifically, the septum 43 is seated within an annular recess 44 formed in a proximal face of the housing 31. An annular collar 45 is then formed over the housing 31, the annular collar 45 abutting the annular septum recess 47 and thus retaining a configuration of the septum 43. The annular collar 45 may be formed of a shape and size to circumferentially abut the outer surface area of the housing 31 and may be held in place using a means known in the art.

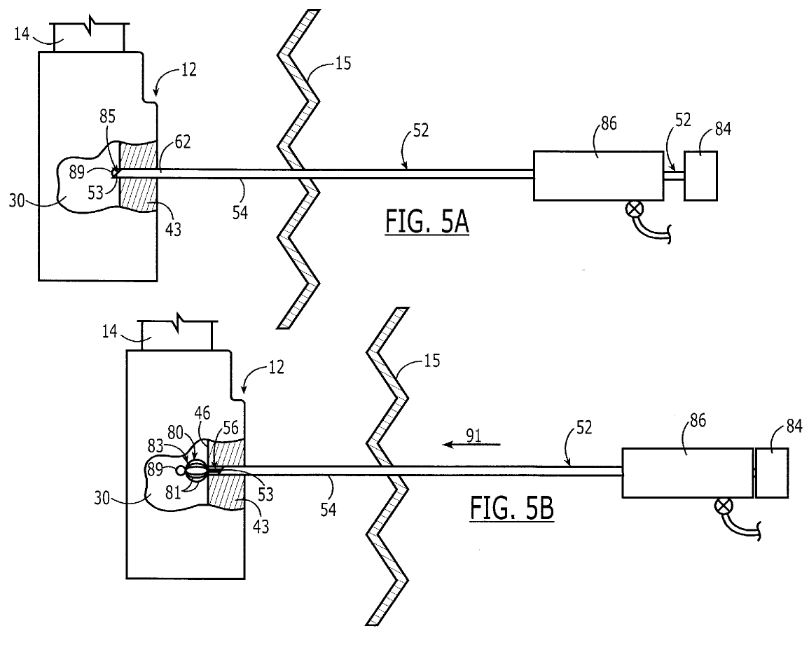

Referring to FIGS. 4A-4C, the needle 52 includes an elongated cannula 54 extending distally out of a handle assembly 82. The cannula 54 includes an elongate shaft 58 extending from a proximal end 60 adjacent the handle assembly 82 to a distal end 62 comprising a tissue penetrating tip 68, which allows the needle 52 to be more easily introduced through tissue while minimizing tissue trauma. A lumen 64, shown in greater detail in FIG. 4C extends through the length of the shaft 58 to a distal opening 53. The lumen 64 is sized and shaped to slidably receive a probe 56 therein while remaining in fluid communication with the distal opening 53. As will be described in greater detail below, the probe aids in preventing coring of the septum 43 while serving to anchor the needle 52 in place in the reservoir 30. In this manner, fluids may be infused or withdrawn from the reservoir 30 into which the distal end 62 is open while the probe is inserted therein. In an alternate embodiment, the cannula 54 may be formed with dual-lumens to maintain separate channels for each of the probe 56 and the fluids to be delivered and/or withdrawn.

The shaft 58 of the cannula 54 may be composed of any material known in the art as suitable for forming needles for power injection. For example, the shaft 58 may be formed of any known suitable substantially rigid, metal or plastic material, such as, for example, stainless steel. The shaft 58 will have a length similar to that of known power injection needles. For example, the shaft 58 may typically have a length in the range from 5 cm to 30 cm, and more preferably, from 10 cm to 25 cm. An outer diameter of the shaft 58 is consistent with its intended use as a non-coring needle and is preferably in the range of 0.7 mm to 5 mm, and, more preferably, from 1 mm to 4 mm.

The probe 56 is slidably disposed within the lumen 64 and comprises a probe shaft 79 extending from a proximal end 76 thereof to a distal end 78 comprising a non-coring obturator 85. A self-expanding anchoring mechanism 80 is coupled to the probe shaft 79 proximally of the obturator 85 and substantially adjacent thereto. The probe shaft 79 may, for example, be formed of a suitably rigid material, so that it has the required axial strength to slide within the lumen 64 and move the obturator 85 and the anchoring mechanism 80 into and out of the shaft 58. In an exemplary embodiment, the probe shaft 79 is composed of stainless steel.

The obturator 85 is sized and shaped to facilitate the anti-coring properties of the needle 52. In particular, the obturator 85 includes a spherical tip coupled to the probe shaft 79. The obturator 85 generally may be formed, for example, of any suitable substantially rigid, metal or plastic as those skilled in the art will understand. The obturator 85 is preferably sized to be slidably received in and to closely fit the lumen 64 so that it may be slid in and out of the lumen 64 and so that, when received therewithin, it substantially seals the lumen 64 minimizing coring of the septum 43

The self-expanding anchoring mechanism 80 in this mechanism comprises a resilient basket structure including an array of individual splines 81, opposite ends of which are connected to the distal end 78 of the probe shaft 79. Each of the individual splines 81 comprises, for example, a spring wire formed from a metal having a suitable shape memory, such as stainless steel, nickel-titanium alloys, spring steel alloys, and the like. Alternatively, the splines 81 may be formed from other material that will retain a memorized shape as known to those of skill in the art. The curved proximal ends of the splines 81 are shaped so that they will assume a radially constrained configuration as they are drawn into the lumen 64 of the cannula 54 and will return to the memorized radially divergent configuration when axially extended from the cannula 54.

The handle assembly 82 comprises a handle member 84 mounted to the proximal end 76 of the probe shaft 79, and a handle sleeve 86 mounted to the proximal end 60 of the cannula 54. The handle member 84 is slidably engaged with the handle sleeve 86 (and the cannula 54). The handle member 84 and handle sleeve 86 can be composed of any suitable rigid material, such as, e.g., metal, plastic, or the like. The handle assembly 82 also includes an infusion port 88 mounted within the handle sleeve 86. The infusion port 88 is in fluid communication with the lumen 64 via a handle sleeve lumen (not shown) formed in the handle sleeve 86 and is sized and shaped to mate with a distal end of the tube 92 of the power injection pump 72. The handle sleeve lumen (not shown) may also include a valve (not shown) for maintaining a fluid-tight seal of the handle sleeve lumen.

It may be readily appreciated that longitudinal translation of the probe 56 relative to the cannula 54 in a proximal direction 93 can be achieved by holding the handle sleeve 86 and displacing the handle member 84 in the proximal direction 93, thereby retracting the anchoring mechanism 80 into the distal end 62 of the cannula 54 and thus retracting the spherical tip 89 of the obturator 85 proximally to seal the outlet port 53 (FIG. 4A). The retracting of the obturator tip 89 and the anchoring mechanism 80 configures the fluid injection needle 52 into an insertion/removal state. Because the splines 81 are formed of spring steel, they may be drawn within the cannula 54, during percutaneous insertion.

In contrast, longitudinal translation of the probe 56 relative to the cannula 54 in a distal direction 91 can be achieved by holding the handle sleeve 86 and displacing the handle member 84 in the distal direction 91, thereby deploying the obturator 85 and the self-expanding anchoring mechanism 80 from the distal end 62 of the cannula shaft 58 (FIG. 4B). The deployment of the obturator 85 and the self-expanding anchoring mechanism 80 from the distal end 62 configures the fluid injection needle 52 into the fluid delivery/withdrawal state. The deploying of the anchoring mechanism 80 distally from the cannula 54 frees the anchoring mechanism 80 to assume its radially divergent shape.

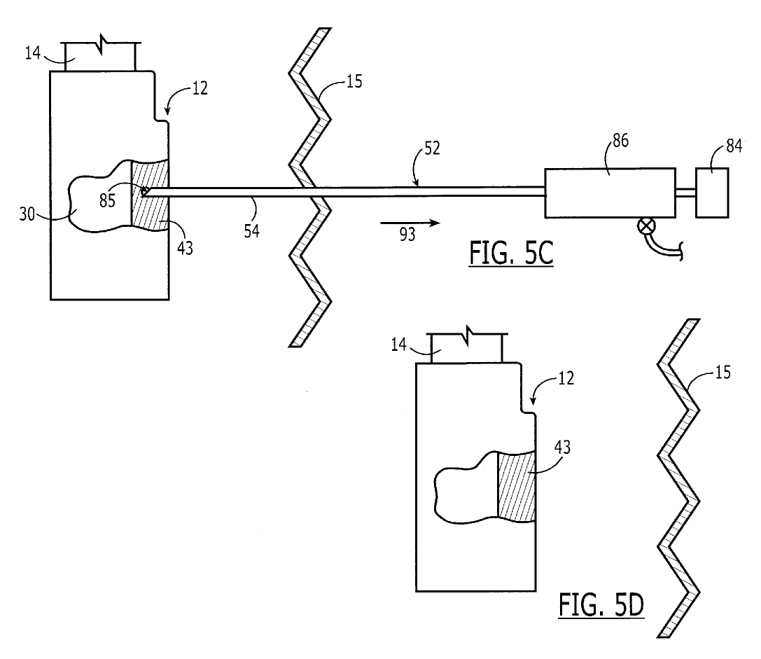

Referring now to FIG. 5A-5D, a method of injecting a medication or fluid into the subcutaneous port 12 using the fluid injection needle 52 is shown. First, the septum 43 of the subcutaneous port 12 is located beneath the skin 15 of a patient, e.g., by pushing with a finger or other device, and as shown in FIG. 5A, the injection needle 52 is inserted through the skin 15 and through the septum 43 of the inlet port 42, so that a distal end 62 of the needle 52 lies at or within the reservoir 30. The injection needle 52 is inserted through the septum 43 while the needle 52 is in the insertion/removal state; that is, the obturator 85 is located within distal end 62 of the cannula 54 with the spherical tip of the obturator 85 seated within the outlet port 53. Significantly, the spherical tip of the obturator 85, when seated within the outlet port 53, prevents the outlet port 53 of the cannula 54 from coring the septum 43.

As shown in FIG. 5B, the injection needle 52 is then placed into its fluid delivery/withdrawal state by deploying the obturator 85 and the anchoring mechanism 80 distally from the distal end 62 of the cannula 54 by advancing probe 56 in the direction of arrow 91; that is, by holding the handle sleeve 86 and displacing the handle member 84 in the distal direction 91. The anchoring mechanism 80 is advanced so that the expanding splines 81 of the resilient basket structure abut an inner surface 46 of the septum 43, thereby stabilizing the injection needle 52 relative to the subcutaneous port 12. Next, a specified amount of the medication is injected into the reservoir 30 by the power injection pump 72 (shown in FIG. 1) through the injection needle 52, which medication flows through the catheter 14 via the outlet port under pressure and into the venous system.

When delivery of the medication has been completed, the injection needle 52 is placed into its insertion/removal state by retracting the anchoring device 80 and obturator 85 within the needle cannula 54 in the direction of arrow 93; that is, by holding the handle sleeve 86 and displacing the handle member 84 in the proximal direction 93, as shown in FIG. 5C. Then, the injection needle 52 is withdrawn from the patient, and as shown in FIG. 5D, the septum 43 of the subcutaneous port 12 is left intact.

It may be appreciated that, while the use of the fluid injection needle 52 lends itself well to the delivery of fluid through an implanted subcutaneous port, the fluid injection needle 52 may be used to deliver fluid to other types of implantable devices. For example, the needle 52 can be used to refill the reservoir of a drug pump.

Although particular embodiments of the present invention have been shown and described, it will be understood that it is not intended to limit the present invention to the preferred embodiments, and it will be obvious to those skilled in the art that various changes and modifications may be made without departing from the spirit and scope of the present invention. Thus, the present inventions are intended to cover alternatives, modifications, and equivalents, which may be included within the spirit and scope of the present invention as defined by the claims.

What is claimed is:

1. A device for providing access to an implantable fluid transfer port, comprising:
a needle extending to a distal end insertable into a living body to enter a reservoir of a port implanted therein;
an actuation member extending through a lumen of the needle from an actuator which remains external to a living body accessible to a user thereof;
an obturator coupled to a distal end of the actuation member, an outer diameter of the obturator substantially matching an inner diameter of the lumen of the needle so that, when retracted into the lumen, the obturator seals a distal opening thereof; and
an anchoring mechanism coupled to the actuation member proximally of the obturator, the anchoring mechanism being moved to an expanded state in which an outer diameter of the anchoring mechanism exceeds an outer diameter of the needle when moved out of the lumen of the needle.

2. The device of claim 1, wherein the anchoring mechanism includes a plurality of flexible members biased toward the expanded state so that the anchoring member assumes the expanded state automatically when moved out of the lumen of the needle.

3. The device of claim 2, wherein the flexible members are shaped so that, when the actuation member is withdrawn proximally, the anchoring mechanism is compressed and withdrawn into the lumen.

4. The device of claim 2, wherein the anchoring mechanism, when in the expanded state, forms a substantially spherical basket structure.

5. The device of claim 1, wherein the obturator includes a substantially spherical tip.

6. The device of claim 1, wherein the needle includes a handle on which the actuator is mounted, the handle further including a fluid infusion port in fluid communication with the lumen of the needle.

7. The device of claim 4, further comprising a pump in fluid communication with the fluid infusion port.

8. A method for establishing fluid communication with an internal body structure, comprising:
inserting a needle into a living body and into a port implanted therein while maintaining the needle in an insertion/removal configuration, the needle comprising an obturator which, in the insertion/removal state, seals a distal opening of a lumen of the needle and an expandable anchoring mechanism which, in the insertion/removal state, is compressed within the lumen of the needle proximal of the obturator;
moving the needle into a fluid transfer state in which the obturator is extended distally from the distal opening and the anchoring mechanism is extended distally out of the lumen of the needle to assume an expanded configuration to engage a structure of the port and retain the needle in a desired position therein; and
transferring fluid between the internal body structure and the needle.

9. The method of claim 8, wherein the fluid is injected into the fluid transfer port at a rate of approximately 5 cc/sec.

10. The method of claim 8, wherein the port includes a self sealing septum through which the needle is inserted into the port, further comprising the step of engaging an inner surface of the septum with the anchoring mechanism before transferring fluid between the needle and the internal body structure.

11. The method of claim 8, further comprising returning the needle to the insertion/removal state after the fluid transfer is complete and withdrawing the needle from the port and the body.

12. The method of claim 8, wherein the needle is moved between the insertion/removal state and the fluid transfer state by operating an actuator coupled to the obturator and the anchoring mechanism via an actuation member extending through the lumen of the needle.

13. The method of claim 8, wherein the needle is a power injection needle and wherein, fluid is supplied to the needle by a pump for power injection to the internal body structure.

14. The method of claim 8, wherein in the expanded configuration the anchoring mechanism forms a substantially spherical basket.

15. The method of claim 14, wherein the anchoring mechanism includes a plurality of flexible members biased to assume the expanded state.
